(19) United States
(12) Patent Application Publication
(10) Pub. No.: US 2004/0156934 A1
(43) Pub. Date: Aug. 12, 2004

(54) Breast mold

(76) Inventor: Antonio Luis Francalacci Franca, Curitiba (BR)

Correspondence Address: Nicholas P. Chiara, Esq., HEDMAN & COSTIGAN, P.C., 1185 Avenue of the Americas, New York, NY, 10036, US

(21) Appl. No.: 10/772,708

(22) Filed: Feb. 5, 2004

(30) Foreign Application Priority Data
Feb. 7, 2003 (BR) MU 8300145-0

(51) Int. Cl. A61F 2/52; B29C 33/00

(52) U.S. Cl. 425/2; 623/7

(57) ABSTRACT

BREAST MOLD is a laminate plastic object, rigid and transparent, thermally molded having a cavity with the geometrical shape and size as equal as possible to a human breast.

### FIELD OF THE INVENTION

It relates to an object of practical usage made of laminate plastic, rigid and transparent, thermally molded with a cavity having a geometrical shape as equal as possible to a human breast.

### BACKGROUND OF THE INVENTION

It is possible to implant breast prosthesis by means of plastic surgery and also to mold a bosom with different geometrical shapes and sizes. In this case of surgical process the expected outcome doesn't depend only on the professional's technical knowledge or skill but also on the client's plastic desired shape. While in the reconstructive surgery the surgeon has all the ethic-professional autonomy, in the cosmetic plastic surgery he's hired to perform a definite task in order to satisfy the contractor's desires and necessities. The understanding between surgeon and client must be clear and detailed in order the expected outcome be equal or almost equal to what had been agreed during the client's first visit. The lack of a clear definition of what had been really agreed has caused a lot of trouble either to the patient as to the surgeon. Sometimes it happens that after a breast prosthesis implantation is done the patient gets stunned with the bosom size, doubting what had been agreed previously with the surgeon. These situations of constraint and discomfort in a surgeon and patient relationship may be avoided by using “breast molds”, an object of practical usage hereby described.

### SUMMARY OF THE INVENTION

The breast mold hereby described is a piece of rigid material such as a molded plastic with a cavity having a geometrical shape and size like a female breast. As the female breast has different sizes according to women's anatomy, it has been established a set of plastic molds being their sizes compatible with the breasts volume usually found.

The breast molds have the function of detailing the surgeon X patient understanding and to make better explicit the expected outcome in the prosthesis implantation. The previously selected mold is than the service render contract documental evidence between the surgeon and the patient.

### BRIEF DESCRIPTION OF THE DRAWINGS

FIG. 1 is the rigid plastic mold side cut view showing the transparent wall (1), cavity (2), contour (3) and the nipple (4).

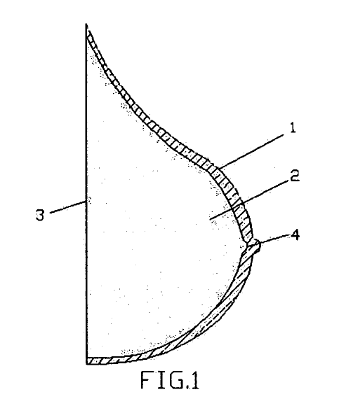

FIG. 2 is the rigid plastic mold front view showing the transparent wall (1), contour (3) and the nipple (4).

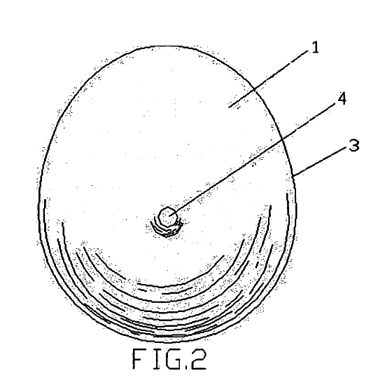

### DETAILED DESCRIPTION OF THE INVENTION

The breast mold is made from a transparent thermally deformable plastic plate. The first step is to prepare the breast models in rigid pieces equal in shape and size to the desired breast. Just after the plastic plate is molded over the breast model providing than the breast mold. The mold wall (1) is transparent, being this technical characteristic essential to check the breast after the prosthesis implantation and to proceed the effective expected outcome comparison.

1- BREAST MOLD, characterized by a transparent wall (1) made of a rigid transparent plastic plate, molded so as to form a cavity (2) and the said cavity (2) having the geometric shape of a human breast.
